(19) United States
(12) Patent Application Publication
(10) Pub. No.: US 2011/0172775 A1
(43) Pub. Date: Jul. 14, 2011

(54) INTERBODY IMPLANT WITH GRAFT RETAINING BONE CAP

(76) Inventors: Eric Flickinger, Atlanta, GA (US); Stephen B. James, Cumming, GA (US); Adam Sclafani, Uniontown, OH (US)

(21) Appl. No.: 12/987,002

(22) Filed: Jan. 7, 2011

Related U.S. Application Data
(60) Provisional application No. 61/293,021, filed on Jan. 7, 2010.

(51) Int. Cl. A61F 2/44

(52) U.S. Cl. 623/17.16

(57) ABSTRACT

Systems and methods for retaining bone graft material in an interbody implant system are provided. In one embodiment of the invention, there is provided an interbody implant system comprising an implant device and at least one sliding bone cap device. The implant device includes a body defining a cavity for holding bone graft material therein and a plurality of fenestrations on at least one surface of the body to allow bone to grow through the body of the implant. The implant is connectable to at least a portion of the at least one sliding bone cap, wherein once connected, the at least one sliding bone cap prevents the bone graft material from being expelled from the body of the implant during insertion of the implant into the interbody disc space.

### RELATED APPLICATION

This application claims the benefit of U.S. Provisional Patent Application No. 61/293,021, entitled “Interbody Implant with Sliding Bone Cap,” which was filed on Jan. 7, 2010, the contents of which are incorporated herein by reference.

### FIELD OF THE INVENTION

This invention relates generally to surgical implants for use in spinal surgery and, in particular, to an improved surgical implant system for bone grafting.

### BACKGROUND

Degenerative disc disease is typically caused by a loss of disc space height, leading to a narrowing of the neural foramen and subsequent neural compression, and causing back and radicular pain. Instability of the posterior elements can lead to conditions such as spondylolisthesis or spinal stenosis. In the case of spondylolisthesis, a vertebral body slips forward in relation to an adjacent vertebrae. This movement of the vertebral body narrows the foramen and results in painful pressure on the nerve roots. In the case of spinal stenosis, the spinal canal narrows and compresses the spinal cord and nerves.

Degenerative disc disease may often be resolved through a spinal fusion procedure using an interbody implant (one which is implanted between the bodies of two adjacent vertebrae). Interbody implants have been used widely since the mid 1930s to aid in spinal fusion. Such interbody implants may be formed from titanium, carbon fiber, allograft, or other suitable material including, but not limited to, biocompatible materials such as the Paek Plastics family. Implantation of a substitute graft is designed to reestablish normal disc height, provide immediate stability to the motion segment, and provide a matrix for fusion of the implant with the patient's natural bone structures. Bone tissue is capable of regeneration and will grow if adequate space is provided. Therefore, when the patient's bone grows into the implant device, the fusion becomes solid and movement is eliminated at that level.

Typically, an open implant device is filled with a graft material and placed inside the disc space. Such graft material may come from the patient's own body. Alternatively, the graft material may be any suitable artificial, synthetic, or natural substitute. Once the implant containing the graft material is properly placed in the disc space, a biological reaction is triggered, which results in bone growth. Over time, as the patient's native bone begins to grow, the natural bone will replace the graft material, resulting in new bone located in the target region of the spine.

The interbody space for lumbar surgery has always challenged surgeons when trying to access the space to achieve arthrodesis. Multiple surgical methods have been employed to place the interbody implant into the disc space: a posterior approach (posterior lumber interbody fusion—PLIF), a transforaminal approach (transforaminal lumbar interbody fusion—TLIF), an anterior approach (anterior lumbar interbody fusion—ALIF) or a direct lateral approach (extreme lateral interbody fusion—XLIF).

Proper distraction during a PLIF procedure must be achieved in order to gain compression of the implant through ligamentous taxis. Proper distraction allows natural compression across the disc space via the annulus and other posterior elements as well as the anterior longitudinal ligament. This compression delivered to the implant helps stabilize the implant, which prevents expulsion, and keeps the grafting material under stress, thus promoting faster fusion and bone healing. Existing techniques for reaching the interbody space from a posterior approach include the use of Cloward dowels, threaded cages, impacted cages and impacted allografts. All of these techniques have limitations as well as complications, as they involve extensive nerve root retraction as well as destabilization through destruction of bony and ligamentous structures.

TLIF involves the removal of one facet joint, usually on the more diseased or symptomatic side of the spine. PLIF is usually performed bilaterally, removing a portion (if not all) of each of the facet joints. Removal of the entire facet joint improves visualization into the disc space, allowing removal of more disc material and insertion of a larger implant. The transforaminal approach limits the nerve root injuries associated with the PLIF procedure because the disc space and spinal canal is approached from one side of the intervertebral space. This allows the surgeon to operate with minimal stretching of nerve roots. Various banana-shaped implants have been designed to be impacted across the disc space to achieve arthrodesis. Although longer, straight implants have been placed across the disc space with some success, the lordotic angle of the spine is harder to properly match with these straight implants. The banana-shaped implant helps maintain proper lordosis when it is placed in the anterior third of the disc space. Despite the benefits of the TLIF procedure, TLIF still suffers from limitations involving bony and soft tissue destruction and bilateral pathology.

ALIF is utilized to avoid the posterior structures of the spine. However, the anterior approach (from the patient's abdomen) to the disc space also presents challenges and limitations because of the potential of vascular injuries. In addition, not all of the lumbar spinal segments can be reached from an anterior incision without potential complications. Retroperitoneal approaches have helped eliminate some of the vascular injuries, but the potential still exists. It is known in the art that revision surgery is greatly complicated by scarring from the initial procedure.

XLIF was devised in an attempt to avoid the complications associated with the posterior and anterior approaches to the spine. This technique provides an additional way to access the interbody space for fusion as well as for motion preservation procedures. XLIF is useful for lumbar fusions from L1-L5 and preserves the entire posterior envelope of the spine. The XLIF procedure can also be performed at levels above the lumbar spine in the thoracic region. XLIF is minimally invasive in that it does not involve cutting of muscle tissue. While there is potential for nerve injury (though limited by using nerve monitoring equipment) and psoas muscle irritation, the muscles are spared through dilation instruments. Once the disc space is exposed, complete discectomy can be performed to prepare the fusion bed. Since the XLIF procedure avoids anterior entry, vascular structures are not compromised or scarred, eliminating possible complications in following salvage procedures. Another drawback of existing systems and techniques for XLIF procedures is that implants are usually undersized from a medial lateral and anterior-posterior approach. When the implant is undersized, and not resting on the cortical edges of the vertebral bodies, they can piston through the softer, interior portions of the vertebral bodies. This can occur with or without endplate sparing techniques.

Each approach has its limitations as well as advantages. From a posterior (PLIF) or transforaminal (TLIF) approach, the individual implants are usually smaller because of the neural structures that prevent access to the total disc space. From an anterior (ALIF) or far lateral (XLIF) approach, the implants are usually quite larger and a fuller, more complete discectomy can be performed without the limitations of retracting neural structures. Thus, larger implants can be utilized that hold more graft material. Regardless of the approach, each implant inserted into the disc space will hold a volume of graft material with the intent of triggering the bone growth biological response.

In existing systems, when the implants are impacted, threaded or placed into the disc space, the graft material can fall out or otherwise become separated from the interbody implant. The expelled graft material may land in undesired or potentially harmful areas of the surgical site, and/or create a nuisance for the surgeon attempting to retrieve the expelled graft material. In addition, if the bone grafting material is a highly concentrated bone morphogenic protein (BMP), it has been documented that BMP can cause ectopic bone formation in unwanted areas if it is expelled from the implant and left in the pathway to the disc space. What is needed is a system for retaining graft material while improving the distraction and bone grafting functions of an interbody implant.

### SUMMARY

The present invention provides an interbody implant system for retaining bone graft. In one embodiment of the invention, there is provided an interbody implant system comprising an implant device and at least one sliding bone cap device. The implant device includes a body defining a cavity for holding bone graft material therein and a plurality of fenestrations on at least one surface of the body to allow bone to grow through the body of the implant. The implant is connectable to at least a portion of the at least one sliding bone cap, wherein once connected, the at least one sliding bone cap prevents the bone graft material from being expelled from the body of the implant during insertion of the implant into the interbody disc space.

In another embodiment of the invention, there is provided an interbody implant system comprising an implant device having a body defining at least one cavity for holding bone graft material therein and at least one retention device. The at least one cavity is configured to receive the at least one retention device, wherein once the at least one retention device is inserted into the at least one cavity, the at least one retention device is securedly connected to the implant and prevents the bone graft material from being expelled from the body of the implant during insertion of the implant into the interbody disc space.

### BRIEF DESCRIPTION OF THE DRAWINGS

FIG. 1 is a perspective view of the interbody implant system according to one embodiment of this invention.

FIG. 2 is a perspective view of the interbody implant system according to one embodiment of this invention.

FIG. 3 is a front view of an interbody implant according to one embodiment of this invention.

FIG. 4 is a front view of the interbody implant of FIG. 3 with the sliding bone cap engaged according to one embodiment of this invention.

FIG. 5 is a perspective view of the interbody implant system according to one embodiment of this invention.

FIG. 6 is a perspective view of the interbody implant system according to one embodiment of this invention.

FIG. 7 is a perspective view of the interbody implant system according to one embodiment of this invention.

FIG. 8 is a perspective view of the interbody implant system according to one embodiment of this invention.

### DETAILED DESCRIPTION

This invention provides an interbody implant system comprising an implant device and a sliding bone cap or retention device. The bone cap or retention device is useful to prevent expulsion of graft material from the implant during insertion of the implant into the interbody disc space. The interbody implant system of this invention is particularly useful for larger implants that may be used in procedures such as ALIF or XLIF, but the inventive interbody implant system may be suitable for implants of any size, shape, or style or for use in various procedures.

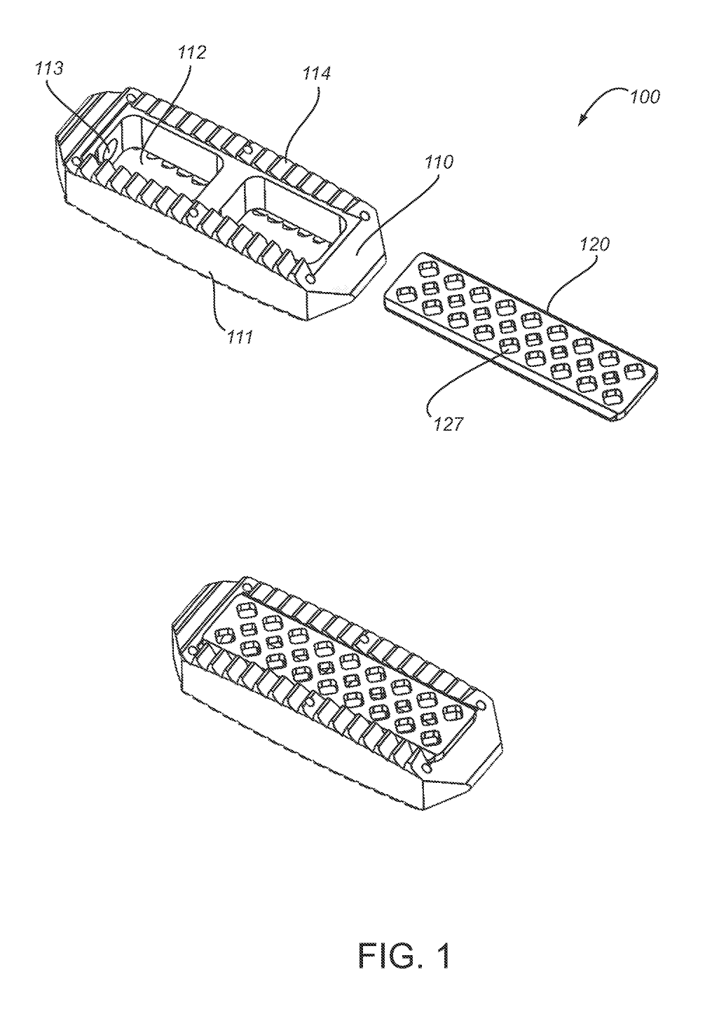

FIG. 1 shows an interbody implant system 100 according to one embodiment of this invention. The interbody implant system 100 comprises an interbody implant device 110 and a sliding bone cap 120. The interbody implant 110 and the sliding bone cap 120 may be constructed from biocompatible metal alloys such as titanium, cobalt-chrome, and stainless steel. The interbody implant 110 and the sliding bone cap 120 may also be constructed from non-metallic materials, including for example, ceramics, resins, or polymers, such as UHMWPE and implantable grade polyetheretherketone (PEEK) or other similar materials (e.g., PAEK, PEKK, and PEK) or even a resorbable polymer. The interbody implant 110 and the sliding bone cap 120 may be constructed of synthetic or natural bone or bone composites. Those skilled in the art will readily appreciate other materials of which the interbody implant 110 and sliding bone cap 120 according to various embodiments of this invention may be composed.

The interbody implant 110 shown in FIG. 1 includes one example of an implant shape, though other shapes and contours may be used. In further embodiments, the interbody implant 110 may include other shapes, such as, for example, a circular shape, kidney shape, semi-oval shape, bean-shape, D-shape, elliptical-shape, egg-shape, or any other shape that would occur to one of skill in the art. In other embodiments, the interbody implant 110 could also be described as being annular, U-shaped, C-shaped, V-shaped, horseshoe-shaped, semi-circular shaped, semi-oval shaped, or other similar terms defining an implant including at least a partially open or hollow construction.

The interbody implant 110 includes a body 111 defining at least one cavity 112. The at least one cavity 112 is at least a partially open or hollow space in the body 111 of the interbody implant 110. The at least one cavity 112 is designed to house bone graft material. The interbody implant 110 further comprises an opening 113 for connecting the interbody implant 110 to an insertion device.

At least one surface of the sliding bone cap 120 may comprise a plurality of fenestrations 127 to allow bone to grow through the interbody implant 110 while retaining bone graft material within the cavity 112 of the implant. The fenestrations 127 may be of different sizes and geometries designed to retain bone graft material during insertion of the interbody implant 110 into the disc space. For example, in certain embodiments, such as that shown in FIG. 1, the plurality of fenestrations 127 may comprise a series of diamond-shaped openings throughout the surface of the sliding bone cap 120. The fenestrations 127 may also comprise a mesh system to house the graft material to keep it from falling out during insertion of the interbody implant 110. In alternative embodiments, other methods, materials, and geometrical fenestrations are used in connection with the sliding bone cap to retain the graft material in the interbody implant.

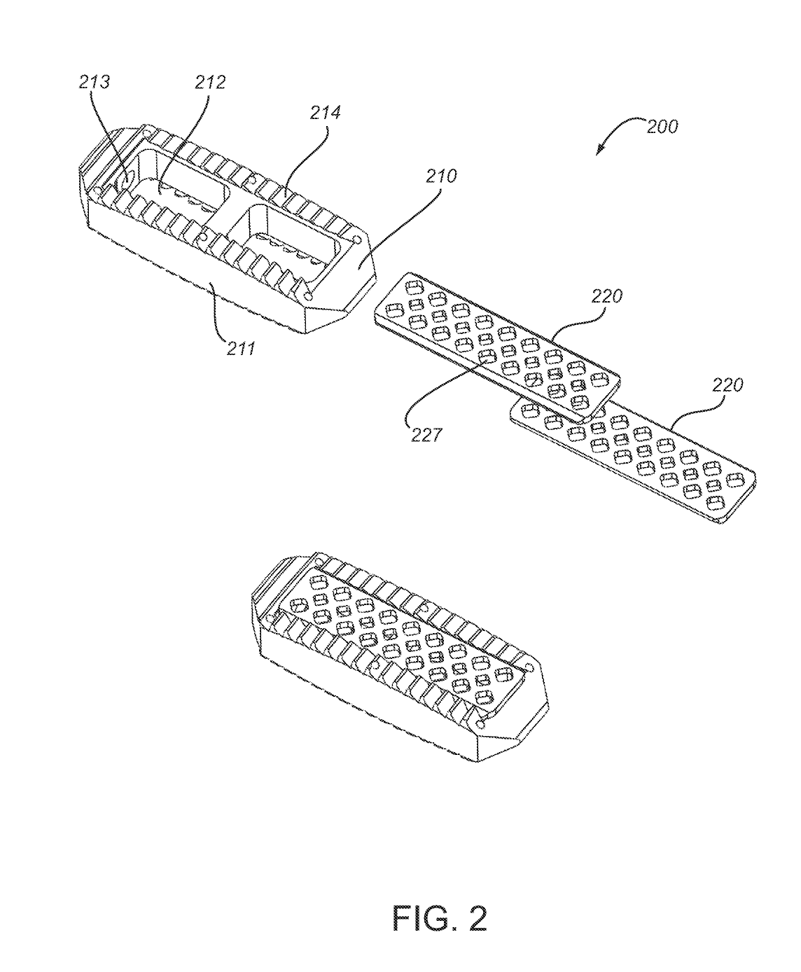

In operation, bone graft material is placed in at least one cavity 112 of the implant 110. Then, the sliding bone cap 120 is connected to the implant by sliding the cap across the at least one cavity 112. Once inserted the sliding bone cap 120 prevents bone graft material from being expelled from the cavity of the implant. As shown in FIG. 2, the interbody implant device 210 may be configured to receive two or more sliding bone caps 220. In this embodiment, once the sliding bone caps 220 are inserted, bone graft material is housed in the cavity 212 of the implant between the two sliding bone caps 220, further preventing the bone graft material from being expelled from the implant during insertion.

The interbody implant 110, 210 used in embodiments of the invention may be designed to ease the distraction and insertion processes of spinal surgery. For example, in certain embodiments, the interbody implant 110, 210 may have a bulleted nose, a rounded nose, rounded surface, or other similar design to aid in the distraction of the disc space during insertion of the implant. Alternatively, the interbody implant 110, 210 may include chamfered or rounded corners to mimic the disc space anatomy and to avoid the neural or vascular structures during insertion into the disc space.

The interbody implant 110, 210 according to embodiments of the invention may additionally or alternatively include a radius to the top, bottom, and/or both sides to mimic the disc space. In other embodiments of the invention, the interbody implant 110, 210 may have at least one rounded side wall to mimic the disc space for a more anatomical fit. The interbody implant 110, 210 may also have a built-in lordotic angle for a more anatomical fit. The interbody implant 110, 210 may contain a rounded surface on the anterior side of the implant to fit into the disc space and allow the denser, cortical edges of the vertebral bodies to rest more anatomically and prevent migration through the endplates of the bodies. Also, the interbody implant 110, 210 can be wider in the anterior-posterior dimension as well as medial lateral dimension to prevent the pistoning through the endplate.

Additionally, in certain embodiments of the invention, the interbody implant 110, 210 may include a toothed pattern 114, 214 on at least one side to prevent migration of the implant once inserted into the disc space. The toothed pattern 114, 214 is also suitable for preventing retro-pulsing out of the disc space, which is a common problem with existing systems. The toothed pattern 114, 214 may comprise angled teeth, castled teeth, parallel teeth, or other rigid surface designs.

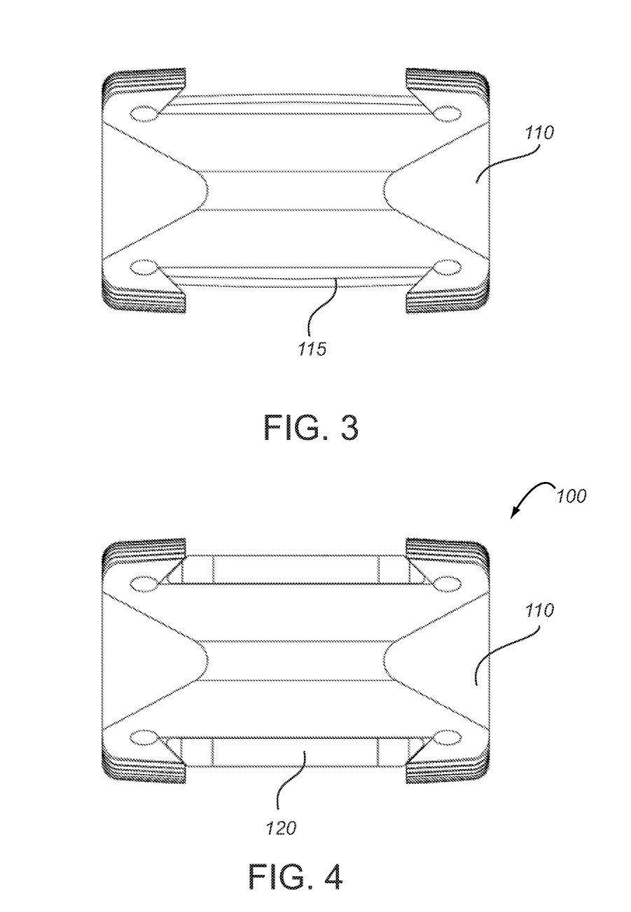

As shown in FIG. 1 and FIG. 2, in certain embodiments of the invention, the sliding bone cap 120, 220 is slideably connected to the interbody implant 110, 210. For example, the interbody implant 110, 210 may include an aperture 115 (shown in FIG. 3) configured to receive the sliding bone cap 120, 220. In this embodiment, the bone cap device 120, 220 is inserted into the aperture 115 of the interbody implant 110, 210 and slid into place. The interbody implant system 100, 200 may include a dovetail design (shown in FIG. 4), which will prevent the bone cap device 120, 220 from disengaging from the interbody implant 110, 210. In certain embodiments, this dovetail design may be squared, rounded, or any appropriate geometry that creates a locking mechanism for preventing the sliding bone cap device 120, 220 from disengaging from the interbody implant 110, 210.

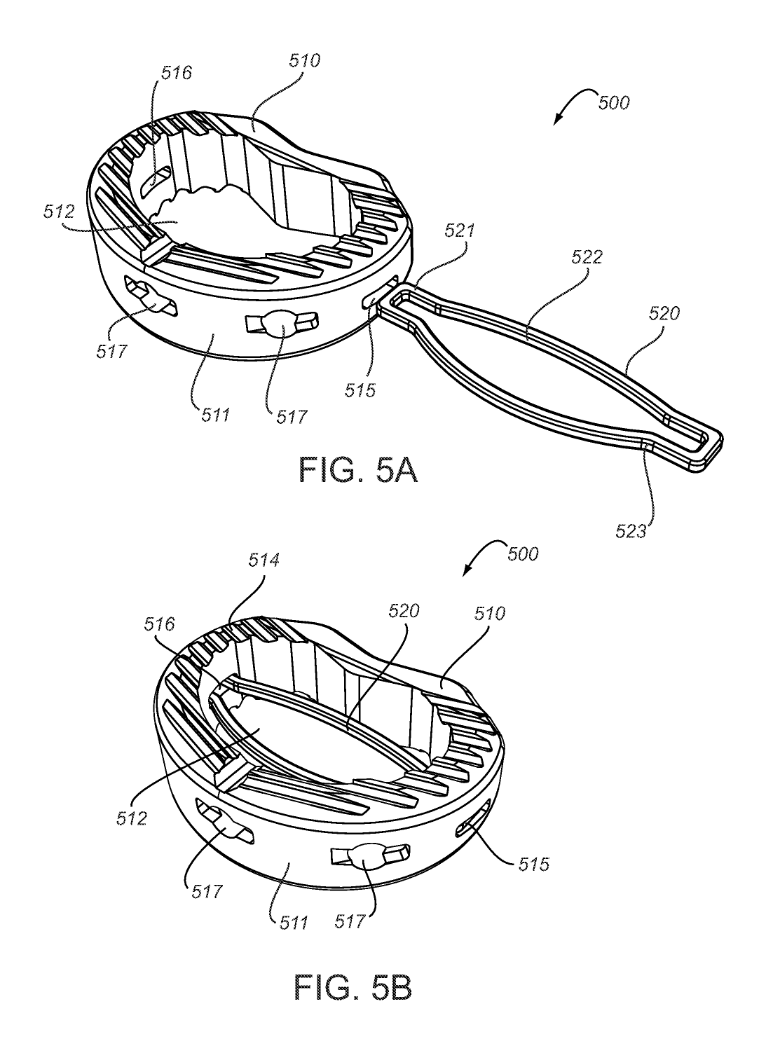

FIG. 5 illustrates an alternative embodiment of an interbody implant system 500. As shown, the interbody implant system 500 includes an interbody implant device 510 and a graft retention device 520. The implant device includes a body 511 that defines a cavity 512 for holding bone graft material therein. The interbody implant 510 includes at least two sides, a top and a bottom, through which bone is able to grow. Alternatively, or additionally, the body 511 of the interbody implant device 510 comprises a plurality of fenestrations 516 through which bone can grow and blood can flow. The plurality of fenestrations 516 in these embodiments of the invention may be different sizes and geometries designed to retain bone graft material during insertion of the interbody implant 510 into the interbody disc space.

The graft retention device 520 comprises a first section 521, a second section 522, and a locking mechanism 523. In certain embodiments, such as that shown in FIG. 5a, the width of the second section 522 is larger than the width of the first section 521. The graft retention device 520 may be constructed in a number of shapes, such as a circular shape, kidney shape, semi-oval shape, bean-shape, D-shape, elliptical-shape, egg-shape, or any other shape that would occur to one of skill in the art. In other embodiments, the bone cap device 520 could also be described as being annular, U-shaped, C-shaped, V-shaped, horseshoe-shaped, semi-circular shaped, semi-oval shaped or any other shape suitable for retaining bone graft material.

As shown in FIG. 5a, the body 511 of the interbody implant device 510 further comprises at least one aperture 515 configured to receive the first section 521 of the graft retention device 520. To engage the interbody implant system 500, the first section 521 of the graft retention device 520 is inserted into aperture 515 and slid into the cavity 512 of the interbody implant device 510 in a longitudinal direction relative to the graft retention device 520 until the first section 521 of the graft retention device 520 mates with locking aperture 516. The locking mechanism 523 of the graft retention device 520 is configured to lock the graft retention device into place once the first section 521 is connected to the locking aperture 516. As shown in FIG. 5b, once connected, the graft retention device 520 is prevented from disengaging from the interbody implant 510. In certain other embodiments, the graft retention device 520 may be prevented from disengaging from the interbody implant 510 simply by friction. Alternatively, the graft retention device 520 may include one or more tabs, pins, or slits in the frame of the cap to provide a stopping or locking point. The one or more tabs, pins, or slits in the frame of the sliding bone cap may also be used to unlock or manually disengage the graft retention device 520 from the interbody implant 510.

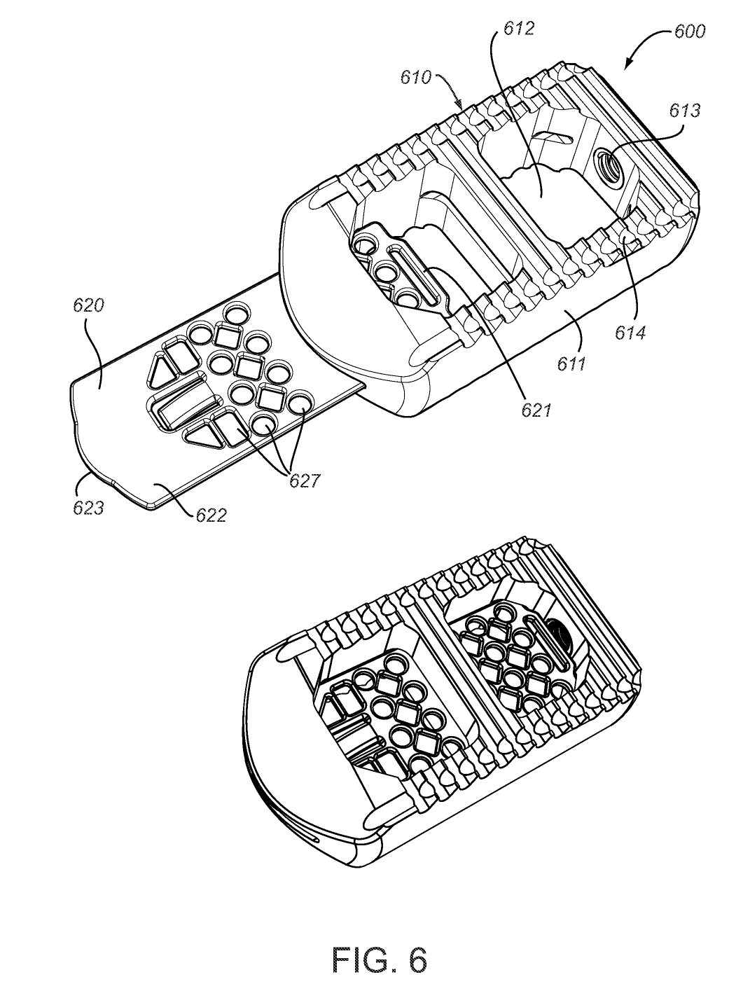

FIG. 6 shows an interbody implant system 600 according to another embodiment of the invention. As shown in FIG. 6a, the interbody implant system 600 includes an interbody implant device 610 with a toothed pattern 614 on at least one side. The interbody implant device 610 is adapted to receive a bone cap device 620. In this and other embodiments, the bone cap device 620 may be a sliding bone cap or other graft retention device. The bone cap device 620 comprises a leading end 621, a surface 622, and a locking mechanism 623. The surface 622 of the bone cap device 620 further comprises a plurality of fenestrations 627 disposed throughout the surface 622 of the bone cap device 620 to allow bone to grow through the interbody implant 610. The fenestrations 627 may be of different sizes and geometries designed to retain bone graft material during insertion of the interbody implant 610 into the disc space. For example, in certain embodiments, the plurality of fenestrations 627 on the surface 622 of the bone cap device 620 may comprise a mesh system to hold in the graft material to keep it from falling out during insertion of the interbody implant 610. In alternative embodiments, other methods, materials, and geometrical fenestrations are used in connection with the sliding bone cap to retain the graft material in the interbody implant.

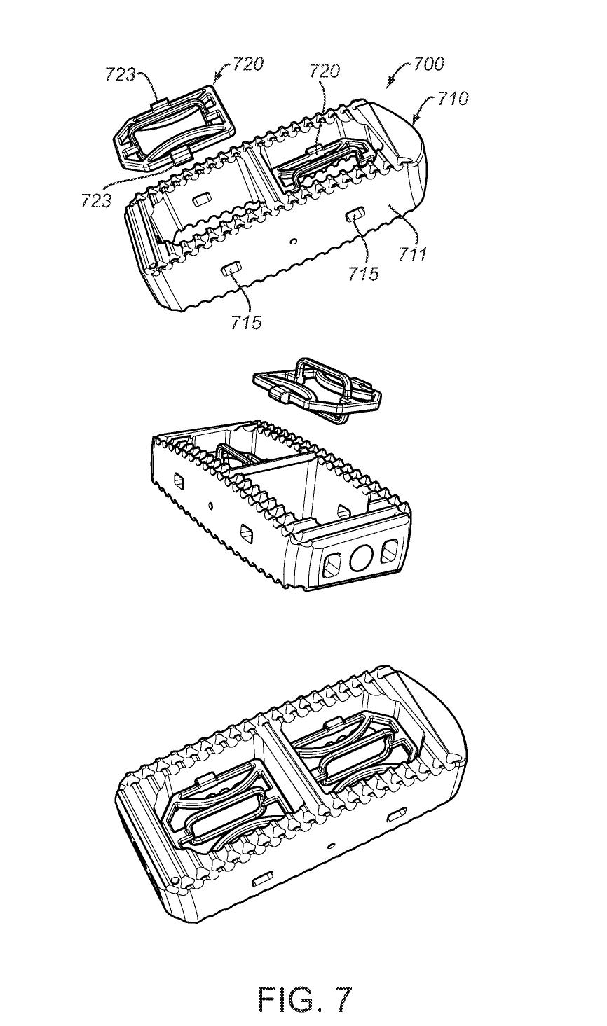

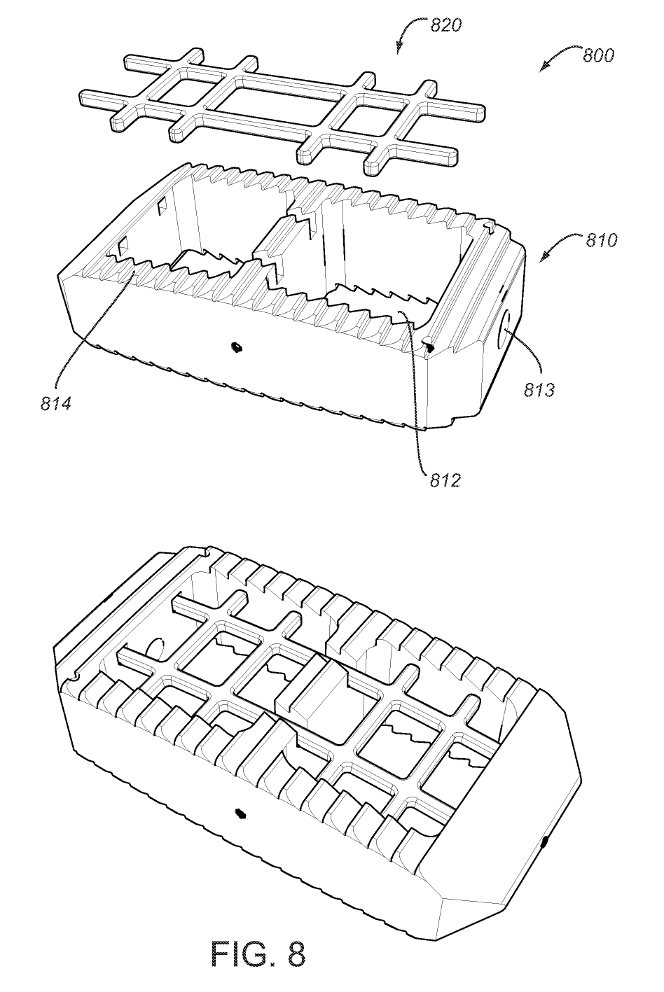

In yet other embodiments of the invention, such as those shown in FIG. 7 and FIG. 8, the bone cap device may be snapped onto or into one or more cavities defined by the body of the interbody implant. For example, in FIG. 7, the body 711 of the interbody implant 710 defines two cavities 712. The bone cap device 720 is a snapping bone cap with locking tabs 723 configured to correspond to apertures 715 in the body 711 of the interbody implant 710. To engage the interbody implant system 700, the bone cap device 720 is squeezed such that the locking tabs 723 are moved in the direction of each other. The bone cap device 720 is then inserted into the cavity 712 of the interbody implant 710 so that the locking tabs 723 are aligned with the apertures 715. When the bone cap device 720 is released, the locking tabs 723 are received by corresponding apertures 715, locking the bone cap device 720 into the cavity 712 of the interbody implant 710.

FIG. 8 shows another interbody implant system 800 according to one embodiment of the invention. The interbody implant system 800 comprises an implant device 810 and a bone cap device 820. In this and other embodiments, the bone cap device 820 is a lattice. The lattice 820 is insertable into the cavity 812 of the implant device 810. The inner surface of the implant device 810 includes openings for receiving at least a portion of members from the lattice 820. The cavity 812 is further configured to receive and securedly connect to the lattice 820 when the lattice is inserted into the cavity as shown in FIG. 8b. The lattice 820 may comprise members organized in a perpendicular fashion as shown in FIG. 8a. Alternatively, the lattice 820 may comprise members intersecting at angles to form diagonally shaped openings in the lattice. Those skilled in the art will appreciate other design arrangements for the lattice 820 in order to retain bone graft material in the implant.

As can be seen in FIG. 7 and FIG. 8 and can be appreciated by those skilled in the art, the bone cap device according to various embodiments of the invention may comprise a single member or multiple separate pieces. In some embodiments of the invention, the multiple pieces that comprise the bone cap device may be of different sizes and shapes. The bone cap device may be placed in one cavity, both cavities, or multiple cavities of the interbody implant, depending on the size, shape, and style of the implant. The implant may or may not include an I-beam shaped structure or reinforcing web depending on the strength of the material.

Additionally, in certain embodiments of the invention, one or more pieces of the bone cap may include a toothed pattern on at least one side to prevent migration of the implant once inserted into the disc space. The toothed pattern may comprise angled teeth, castled teeth, parallel teeth, or other rigid surface designs.

Alternatively, the bone cap may fit under a ledge of the interbody implant or mate up to the ledge of the interbody implant. In addition to the embodiments described above, those skilled in the art will readily appreciate other means for connecting the interbody implant with the bone cap, each of which is contemplated by the present invention.

Based on the foregoing, it can be seen that the present invention provides an interbody implant system for retaining bone graft material. Many other modifications, features and embodiments of the present invention will become evident to those of skill in the art. It should be appreciated, therefore, that many aspects of the present invention were described above by way of example only and are not intended as required or essential elements of the invention unless explicitly stated otherwise. Accordingly, it should be understood that the foregoing relates only to certain embodiments of the invention and that numerous changes may be made therein without departing from the spirit and scope of the invention as defined by the following claims. It should also be understood that the invention is not restricted to the illustrated embodiments and that various modifications can be made within the scope of the following claims.

1. An interbody implant system comprising:
an implant device including
a body defining at least one cavity for holding graft material therein; and
a plurality of openings on at least one side to allow bone to grow through the implant device;
at least one bone cap device comprising a plurality of openings on at least one side to allow bone to grow through the implant,
wherein the implant device is connectable to at least a portion of the at least one bone cap device and wherein once connected, the bone cap device prevents the graft material from being expelled from the implant during insertion of the implant into the disc space.
